(19) United States
(12) Patent Application Publication
(10) Pub. No.: US 2002/0193371 A1
(43) Pub. Date: Dec. 19, 2002

(54) Drugs which can be used in the treatment of cancer

(76) Inventors: Adam Telerman, Paris (FR); Robert Amson, Paris (FR); Marius Tuijnder, Mechelen (BE)

Correspondence Address: PENNIE AND EDMONDS, 1155 AVENUE OF THE AMERICAS, NEW YORK, NY, 100362711

(21) Appl. No.: 09/885,031

(22) Filed: Jun. 20, 2001

(30) Foreign Application Priority Data
Jun. 1, 2001 (FR) 01/07285

(51) Int. Cl. A61K 31/5415; A61K 31/495

(52) U.S. Cl. 514/225.8; 514/255.04

(57) ABSTRACT

The present invention relates to the use of a compound inhibiting the expression of the TPT1 gene, or of the products which it controls, for producing a drug which is intended for treating cancer.

The present invention relates to the use of novel classes of compounds which are designed for producing a drug which is intended for treating cancer.

Research carried out by the applicant, in particular in the context of the phenomenon of tumor reversion, has led to the demonstration that certain genes are overexpressed during the tumor phase as compared with the reversion phase.

The overexpression of one of these genes, i.e. the gene TPT1, standing for “translationally controlled tumor protein encoding histamine releasing factor”, demonstrated that the expression of this gene was strongly decreased during tumor reversion.

Thus, a tumor cell line designated U937 is capable of reverting and thereby resulting in what is termed a US cell, that is to say a cell line which no longer exhibits such a pronounced malignant phenotype characteristic.

In the U937 cell line, out of 2,000 sequences, for example, the number of clones for the TPT1 gene was 248 whereas it was no more than 2 in the US cell line.

This has led the applicant to focus on the importance of the histamine activation pathway in the phenomenon of tumor reversion and to demonstrate the activity of products which interfere with this pathway as a way of treating cancer.

More specifically, the present invention relates to the use of compounds which totally or partially inhibit the expression of the TPT1 gene, or of the products which it controls, for the purpose of producing a drug which is intended for treating cancer.

The present invention naturally relates not only to the possibility of inhibiting the expression of the TPT1 gene in the cell but also to that of totally or partially inhibiting the expression of the products whose metabolic chain it controls directly or indirectly, up to and including the release of histamine, in particular.

Among the compounds which can be used within the context of the present invention, those which may more specifically be mentioned are the antihistamines, that is to say the antagonists of the histamine H1 receptors, in particular.

It should be clearly understood that the antineoplastic function of these compounds may be linked to subsidiary elements of the cell, taking into account the fact that, for example, one of the antihistamine prototypes, i.e. polaramine, is inactive within the context of the present invention.

Among these compounds which can be used within the present invention, those which may very particularly be mentioned are the phenothiazine derivatives and the derivatives of the piperazine type.

It is also possible to use other derivatives of the ethanolamine or ethyldiamine type, or else the new generation of antagonists of the histamine H1 receptors which do not have a sedative component.

Those phenothiazine derivatives which may be mentioned are dimethothiazine, hydroxyethylpromethazine, isothipendyl, mequitazine, methdilazine, oxomemazine, promethazine, propiomazine, thiazinamium and trimeprazine.

Those piperazine derivatives which may in particular be mentioned are buclizine, cetirizine, chlorcyclizine, cinnarizine, clocinizine, cyclizine, flumarizine, homochlorcycline, hydroxyzine, meclozine, niaprazine and oxatomide.

Those ethanolamine derivatives which may be mentioned, in particular, are the phenhydramine derivatives such as bromodiphenhydramine and diphenhydramine and their homologues.

The ethylendiamines which may be mentioned are, for example, the compounds of the mepyramine type.

Finally, examples of the various compounds which do not have a sedative component and which may be mentioned are acrivastine, ebastine, tazifyline and terfenadine.

These compounds only represent the basic molecules which can be used in accordance with the present invention; it is also possible to use derivatives, in particular substitution derivatives, of the abovementioned compounds, as well as physiologically acceptable salts.

According to the present invention, treatment of the cancer is essentially understood as being the ability of a compound to selectively destroy the tumor cells without appreciably affecting healthy cells, it being understood that this selectivity can vary depending on the compounds and depending on the condition of the patient and the type of cancer being treated.

As will be observed in the examples which follow, the products according to the present invention exhibit remarkable selectivity and, in particular, a very great ability to destroy tumor cells; in particular, the results have been found to be particularly impressive in relation to cells of the leukemic type and in relation to breast cancer, ductal carcinoma and carcinoma of the mammary glands.

The compounds according to the present invention can be used in the form of pharmaceutical compositions which may be employed by any route of administration; however, in a general manner, preference will be given to using injectable routes, in particular, for treating tumors. It is, of course, possible to use other galenic forms, in particular the oral route. The daily doses have to take account of the compound, the condition of the patient and the nature and stage of the cancer being treated.

The appended results demonstrate that there is a dose above which the product loses most of its activity.

It is also possible to envisage using the compounds according to the present invention in combination with other antineoplastic agents, whether these be antimetabolites, alkylating agents, spindle poisons, intercalating agents or other types of hormonal cytolytic agents or antineoplastic agents, as well as certain proteins such as interferons in accordance with additional chemotherapy processes, with the said compounds being used together or separately in accordance with a protocol which is to be determined for each combination.

The figures represent the results obtained, after 48 hours or 144 hours, by treating either malignant or normal cells with the compounds A, B, C or D. They depict the percentage of surviving cells as a function of a treatment without compound or without product (control) or with a compound in accordance with a dilution as previously indicated.

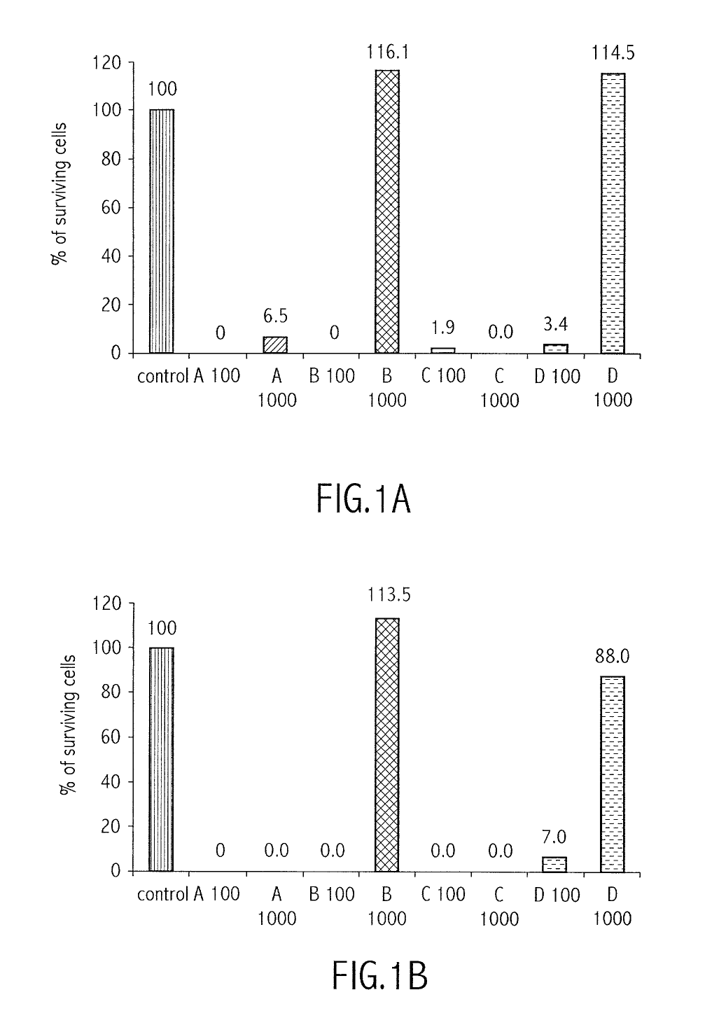

FIG. 1 illustrates the treatment of myeloid leukemia cells, which are derived from the cell line K562, with compound A, B, C or D (1A: at the end of 48 hours, 1B: at the end of 144 hours).

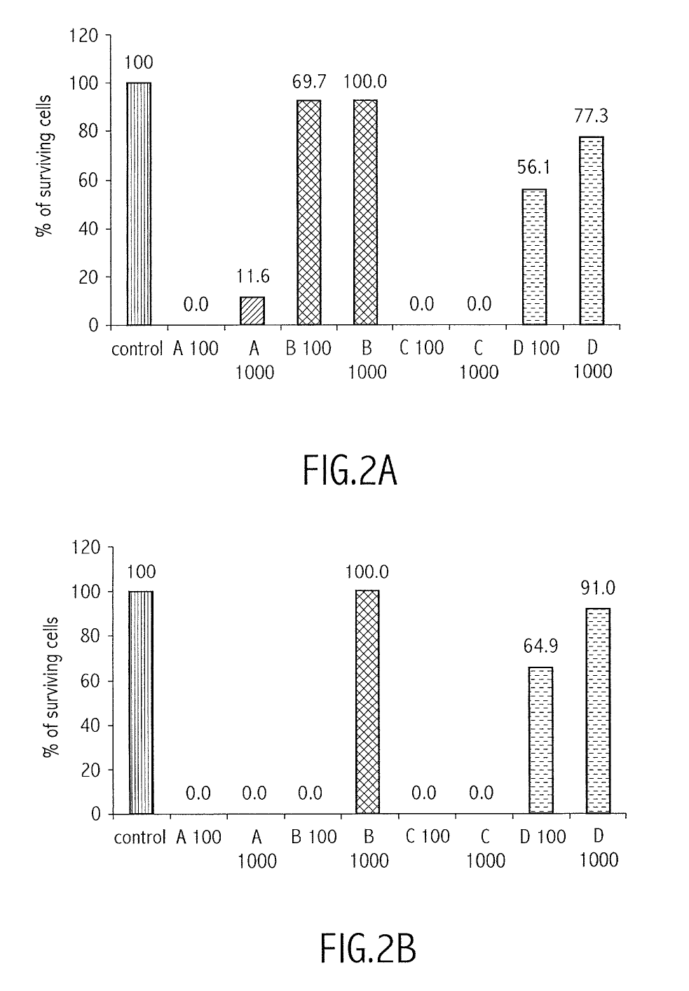

FIG. 2 illustrates the treatment of U937 premonocytic leukemia cells with compound A, B, C or D (2A: at the end of 48 hours, 2B: at the end of 144 hours).

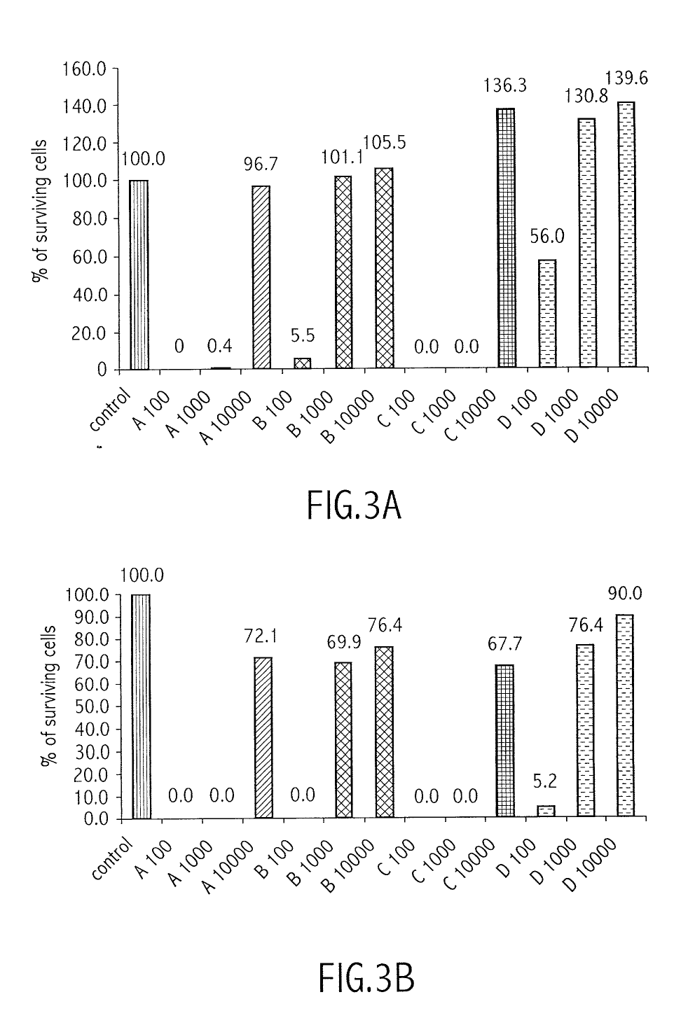

FIG. 3 illustrates the treatment of acute leukemia T cells, derived from the Jurkat cell line, with compound A, B, C or D (3A: at the end of 48 hours, 3B: at the end of 144 hours).

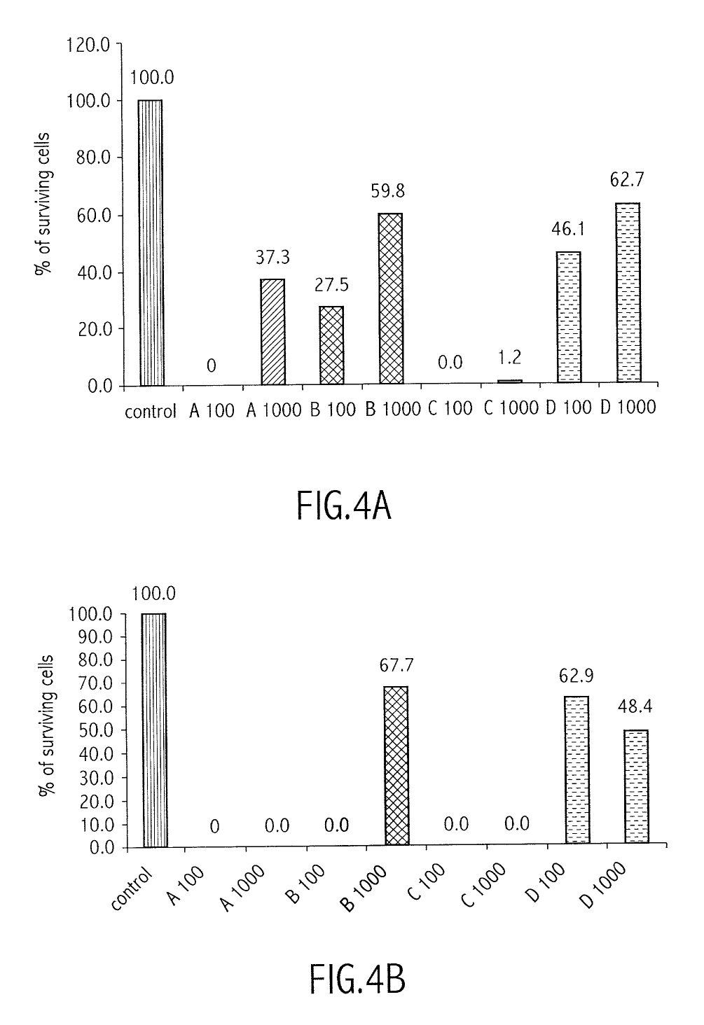

FIG. 4 illustrates the treatment of breast ductal carcinoma cells, derived from the T47-D cell line, with compound A, B, C or D (4A: at the end of 48 hours, 4B: at the end of 144 hours).

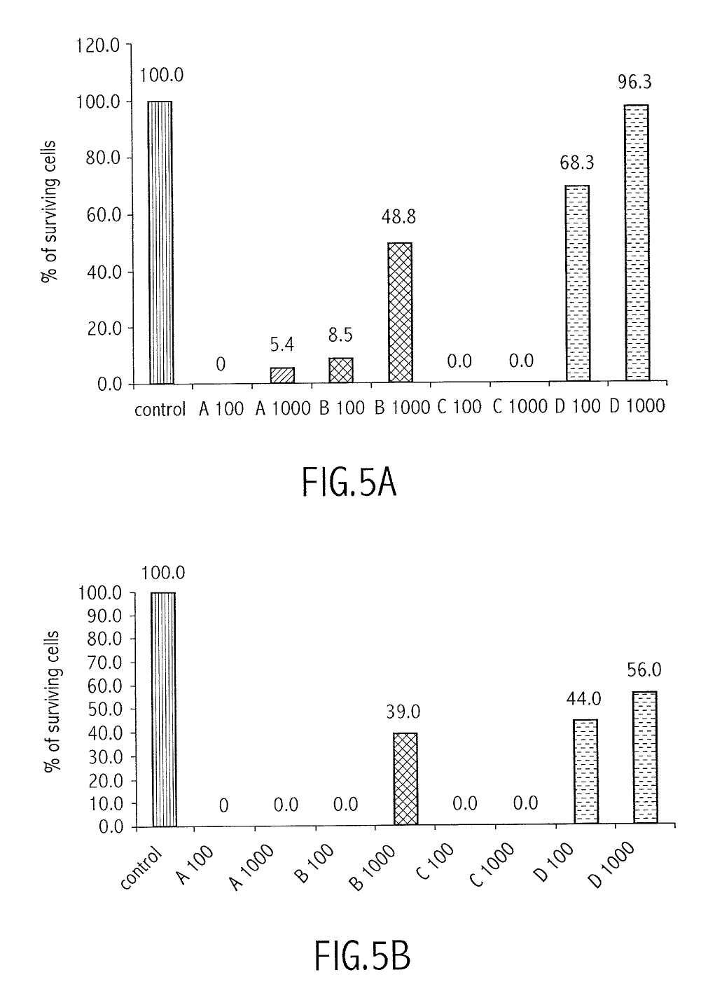

FIG. 5 illustrates the treatment of breast ductal carcinoma cells, derived from the MCF7 cell line, with compound A, B, C or D (5A: at the end of 48 hours, 5B: at the end of 144 hours).

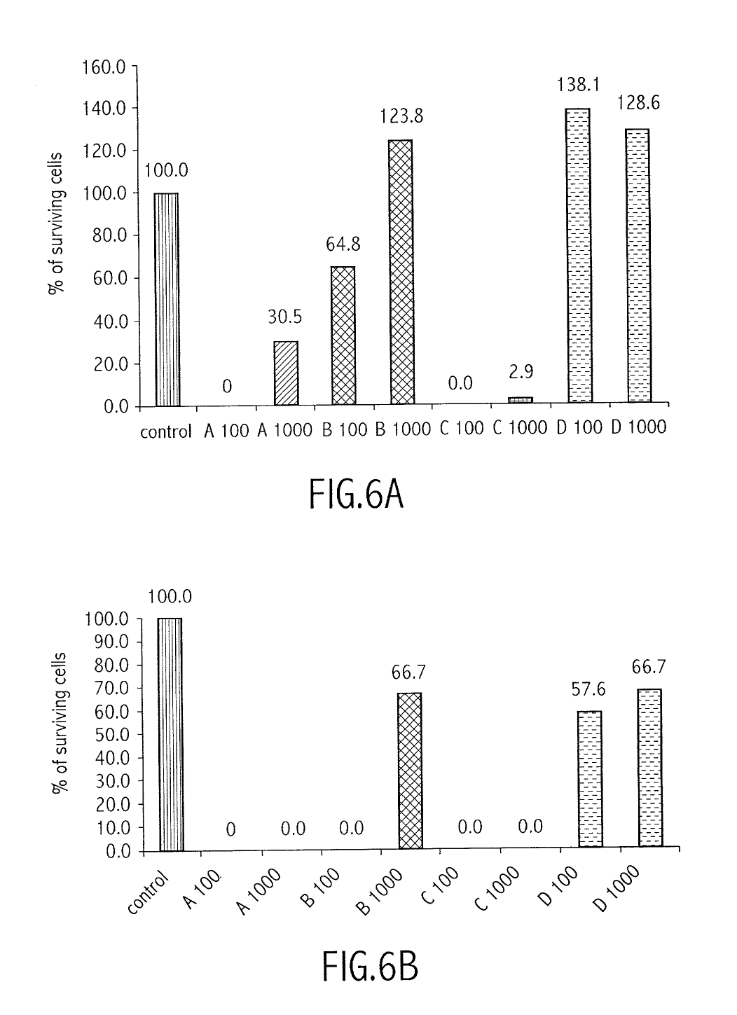

FIG. 6 illustrates the treatment of mammary gland carcinoma cells, derived from the BT20 cell line, with compound A, B, C or D (6A: at the end of 48 hours, 6B: at the end of 144 hours).

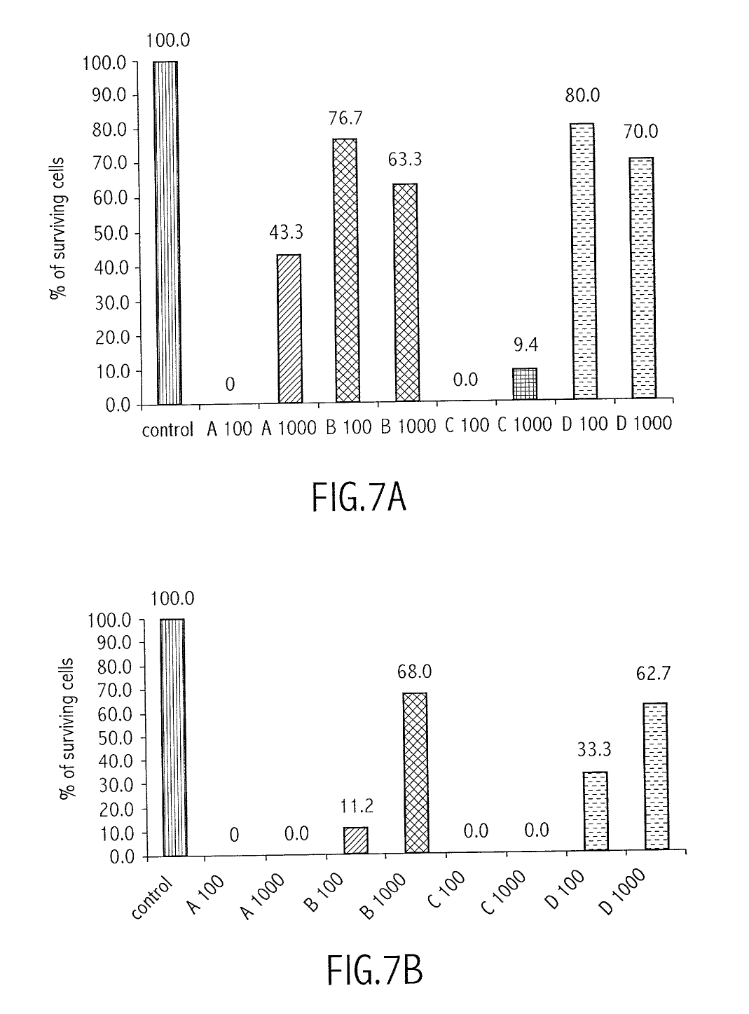

FIG. 7 illustrates the treatment of immortalized, non-tumorigenic breast epithelium cells, derived from the 184B5 cell line, with compound A, B, C or D (7A: at the end of 48 hours, 7B: at the end of 144 hours).

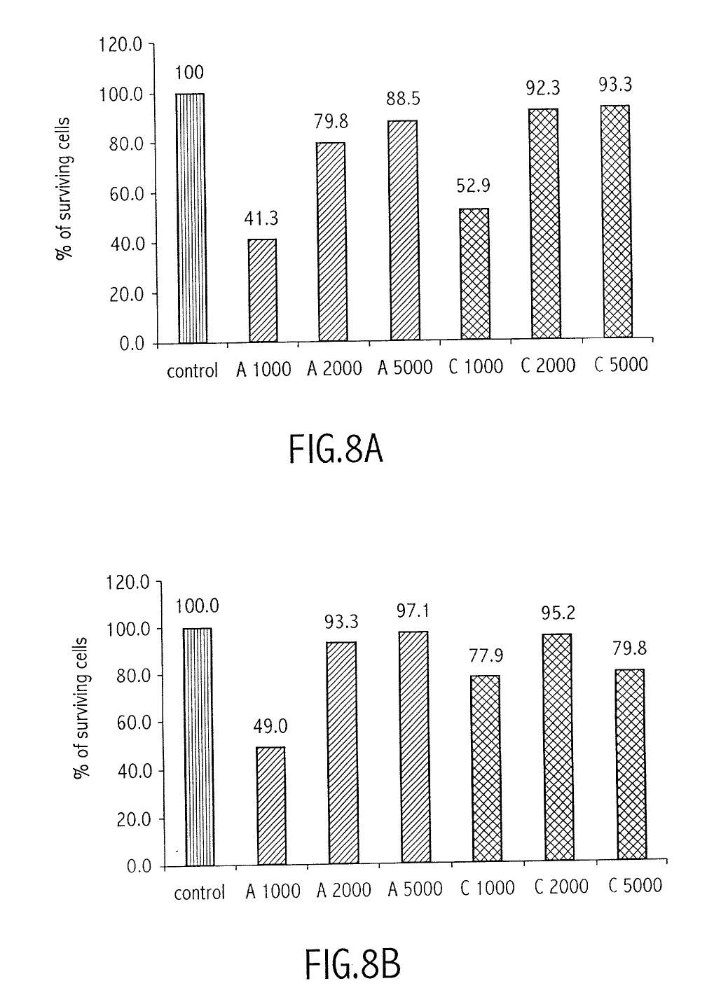

FIG. 8A illustrates the treatment of lymphocytes from donor 1 with compound A or C, at the end of 48 hours.

FIG. 8B illustrates the treatment of lymphocytes from donor 2 with compound A or C, at the end of 48 hours.

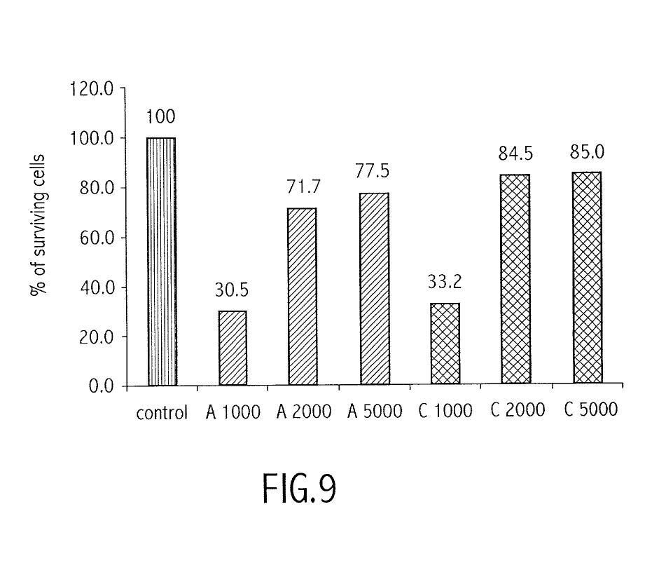

FIG. 9 illustrates the treatment of lymphocytes from donor 3 with compound A or C, at the end of 48 hours.

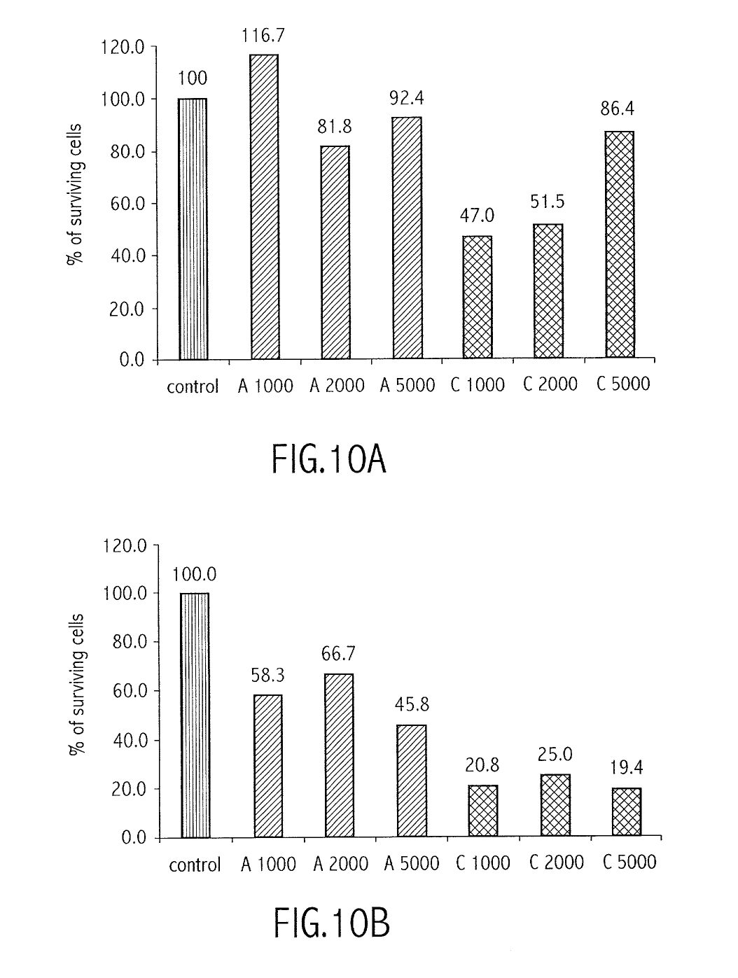

FIG. 10A illustrates the treatment of colorectal adenocarcinoma cells, derived from the LoVo cell line, with compound A or C, at the end of 48 hours.

FIG. 10B illustrates the treatment of immortalized, non-tumorigenic breast luminal epithelium cells, derived from the 184B5 cell line, with compound A or C, at the end of 48 hours.

The examples given below will demonstrate other features and advantages of the present invention.

### EXAMPLES

This study made use of a certain number of human tumors of different origins and of lymphocytes from healthy donors, with these tumors/lymphocytes being treated with varying concentrations of compounds A, B, C and D so as to determine the cytotoxicity of the latter.

These products are:

A) Hydroxyzine dihydrochloride (Atarax® UCB). Solution: 100 mg/2 ml

B) Brompheniramine maleate (Dimegan® FEDERA S.A.). Solution: 10 mg/1 ml

C) Promethazine (Phenergan® Medeva Pharma S.A.). Solution: promethazine hydrochloride: 2,820 g/100 ml

D) Dexchlorpheniramine maleate (Polaramine® Schering-Plough). Solution: 5 mg/1 ml.

These products were added, at different concentrations, to cultures of various malignant cell lines and also to normal cells.

| | |
|---|---|
| K562 | myeloid leukemia |
| KS | revertant of K562 possessing reduced |
| | tumorigenicity |
| U937 | premonocytic leukemia |
| US4 | revertant of U937 possessing reduced |
| | tumorigenicity |
| Jurkat | T lymphocyte, acute leukemia of T cells |
| T47-D | breast cancer, ductal carcinoma |
| MCF7 | breast cancer, ductal carcinoma |
| BT20 | breast cancer, carcinoma of the mammary |
| | glands |
| LoVo | colorectal adenocarcinoma |
| 184B5 | breast, immortalized, non-tumorigenic |
| | cells of the luminal epithelium |
| MCF10A | breast, immortalized, non-tumorigenic |
| | luminal epithelium cells |
| Donors 1, 2, 3 | T and B cells which have been freshly |
| | isolated from 3 healthy donors. |

Leukemic Cell Lines

All the leukemic cell lines were grown and used in logarithmic phase. After 1 day of treatment, the cells were isolated, counted and diluted in a regular growth medium so as to obtain cell densities of 75.10³ cells/ml and 9.375 cells/ml for reading on plates at 48 hours and 144 hours after the treatment, respectively.

In the case of the leukemic cell lines, each dilution product was added to the wells (12 wells per plate, TPP), and 1 ml of cells from the parent solution were added per well after a line had been completed.

All the dilutions of each product were tested 4 times and counted manually and tested with the Alamar reduced assay. The products are diluted in culture medium.

| Control | no product |
|---|---|
| 1:100 dilution | 10 μl of product |
| 1:1000 dilution | 1 μl of product |
| 1:2000 dilution | 50 μl of a 1:100 dilution |
| 1:5000 dilution | 20 μl of a 1:100 dilution |
| 1:10000 dilution | 10 μl of a 1:100 dilution |

Lymphocytes From Healthy Donors

The blood is collected on citrate and diluted 1:1 with 0.15M NaCl. 6 ml of this blood dilution are loaded onto 3 ml of lymphoprep (Nycomed) and centrifuged at ambient temperature for 30 minutes at 800 g. The white cells are isolated and washed with RPMI1640+10% FBS. They are diluted to 450,000 cells/ml in a RPMI1640+10 FBS medium. The same procedure as in the case of the leukemic cells is then followed.

Adherent Cells of the Breast and the Colon

All the cells are grown on their own propagation medium and seeded 24 hours before the products are added. The cells are trypsinized, counted and seeded at 50,000 and 10,000 cells/well in order to read the plates at 48 hours and 144 hours after the treatment.

On the day of the treatment, the medium is replaced (1 ml/well) with the following dilutions:

| Control | no product |
|---|---|
| 1:100 dilution | 100 μl of product in 9.9 ml of |
| | growth medium |
| 1:1000 dilution | 10 μl of product in 10.0 ml of |
| | growth medium |
| 1:2000 dilution | 5 μl of product in 10.0 ml of growth |
| | medium |
| 1:5000 dilution | 2 μl of product in 10.0 ml of growth |
| | medium. |

The product is regarded as being active when the percentage of surviving cells is less than 30%.

The enclosed results show that, when compounds A and C were used at dilutions of from 1:100 to 1:1,000, all the cancerous cells were destroyed, particularly in the case of K562, U937, Jurkat, T47-D, MCF7, BT20 and 184B5, either within 48 hours or, for the most part, within 144 hours.

By contrast, compound B and compound D are found to possess little activity or to be inactive.

Similarly, assays carried out with dilutions greater than 1:1,000, in particular 1:10,000, show that the compound becomes inactive.

The assays which were carried out using the lymphocytes from healthy donors show that the level of survival is very substantial at concentrations of 1:1,000; a differential effect between the lymphocytes from healthy donors and the cancerous cells does therefore exist.

In order to demonstrate that this phenomenon is not linked to a general cytotoxicity, assays were carried out on an LoVo cancer which was resistant to the cytopathic effect to the parvovirus H1. These assays show that the LoVo cancer is totally resistant to the antihistamines.

1. Use of a compound which inhibits the expression of the TPT1 gene, or of the products which it controls, for producing a drug which is intended for treating cancer.

2. Use as claimed in claim 1, characterized in that the compound inhibiting the expression of the TPT1 gene, or of the products which it controls, is an antihistamine.

3. Use as claimed in claim 2, characterized in that the antihistamine belongs to the chemical group of the piperazines and the phenothiazines.

4. Use as claimed in claim 3, characterized in that the product is selected from hydroxyzine and promethazine.

5. Use as claimed in one of claims 1 to 4, characterized in that the cancer being treated is a leukemia or a cancer of the breast.
